(19) United States
(12) Patent Application Publication
(10) Pub. No.: US 2020/0008822 A1
(43) Pub. Date: Jan. 9, 2020

(54) INTRALUMINAL DEVICE

(71) Applicant: RAPID MEDICAL LTD., YOKNEAM, IL (US)

(72) Inventors: Shimon Eckhouse, Haifa (IL); Yuri SUDIN, Modiin (IL); Aharon FRIEDMAN, Haifa (IL); Ronen ECKHOUSE, Shimshit (IL)

(73) Assignee: RAPID MEDICAL LTD., Yokneam, IL (US)

(21) Appl. No.: 16/062,236

(22) PCT Filed: Dec. 16, 2016
(86) PCT No.: PCT/IB2016/002009

§ 371 Date: Jun. 14, 2018

Related U.S. Application Data
(60) Provisional application No. 62/268,284, filed on Dec. 16, 2015.

(51) Int. Cl. A61B 17/221

(52) U.S. Cl. CPC A61B 17/221; A61B 2017/22001; A61B 2017/2212

(57) ABSTRACT

In one exemplary embodiment, an angioplasty device may include a flexible shaft. The angioplasty device may also include an expandable wire mesh structure extending from the flexible shaft. At least one actuator, connected to the expandable wire mesh structure, the actuator being configured to cooperate with the wire mesh structure to transfer angioplasty forces to a vessel obstruction.

### PRIORITY

This application claims the benefit of priority from U.S. Provisional Application No. 62/268,284 filed Dec. 16, 2015, the disclosure of which is herein incorporated by reference in its entirety.

### FIELD

This disclosure relates to intravascular and/or intraluminal medical devices that are configured to retrieve an obstruction from human blood vessels. Obstructions to be retrieved can include clots and clot material.

### SUMMARY

The present disclosure provides for a manually actuatable angioplasty device. In a traditional balloon angioplasty device, a balloon is expanded to exert a force on a stenosis. The balloon compresses the calcified blockage, and during the treatment the balloon may block some or all blood flow through the vessel. In the current disclosure, a wire mesh structure is used to exert forces on a stenosis. The wire mesh may permit greater blood flow through the vessel during the treatment.

Aspects of the present invention permit expansion of the wire mesh angioplasty structure through the exertion of forces on one or more control wires external to a patient's body.

Rather than simply compressing the calcification, with a wire mesh structure, pieces of the blockage can break off and be trapped in the associated wire mesh filter. The angioplasty device can then be at least partially compressed and removed, carrying with it the trapped pieces.

The manually actuated angioplasty device may include a pull wire for exerting radial force. The device may also include multiple pull wires to gain greater radial force. Each pull wire may, for example be connected to a differing portion of the wire mesh structure. The wire mesh structure may be biased closed (compressed) may have no bias at all, or may be biased opened. Once the device is pushed through a catheter, an operator may open the device to the extent needed. To remove, the device may close on its own or may close in response to a reverse actuation force, or may close in response to a combination of such forces. The device may be re-sheathed and removed. Alternatively, if the wire mesh structure has captured portions of a clot and is incapable of being fully re-sheathed, it may be partially re-sheathed, or somewhat compressed, without re-sheathing. This may provide a physician with option of removing clot pieces inside the partially collapsed structure. The device may also include one or more zones configured to apply high radial forces such as may be necessary in an angioplasty procedure. Such forces may cause calcifications to compress, break, or both. If broken, debris may flow into a center of the device, getting caught in a filter of the device. The filter may be located on an upstream side, as a safety measure during removal. A further filter may also be included on the downstream side.

The disclosed embodiments may include an intraluminal device including an elongated structure formed of a plurality of wires. The wires may include groups of woven, or looped wires for structural support. The intraluminal device may include a plurality of sets of looped wires longitudinally located at an intermediate area of the elongated structure. The plurality of sets may be spaced circumferentially about the structure and configured to cooperate with each other to form a plurality of clot entry openings. Openings between wires, or groups of wires may also provide for one or more filters. The one or more filters may be provided at a distal and/or proximal end of the device, for example, and the one or more filters may be configured to assume expanded and compressed positions, individually, or together.

The at least one filter, including at least one grouping of woven wires may be longitudinally located adjacent an intermediate area and may be configured such that when an opening force is exerted on the elongated structure, the at least one grouping may provide structural support to hold open interstices between the plurality of sets of looped wires providing a variable mesh structure for variable radial force. The variable radial force may include a high radial force zone, a very high radial force or high density zone. The device may also include a drug eluting zone.

For example, the adjustable non-blocking angioplasty device may include, for example, a variable mesh structure providing a variable radial force and/or a variable mesh density. In accordance with at least some embodiments in accordance with the present disclosure, the variable mesh structure may correspond to non-uniformity, which may allow for some portions of the device to exert more force than other portions of the device. Also in accordance with at least some embodiments in accordance with the present disclosure, the device may provide for a high radial force zone and a very high radial force or high density zone.

In accordance with at least some embodiments in accordance with the present disclosure, some or all portions of the device may include a drug eluting coating. The coating may be in the middle of the device or may cover the entire device.

In accordance with at least some embodiments in accordance with the present disclosure, the device may for example, a tubular distal filter of varying shapes. (e.g., conical, tubular, etc.) The device may also be provided with a variable mesh density.

In accordance with at least some embodiments in accordance with the present disclosure, the device may include a cover in at least the high radial force zone. The cover may also be drug eluting.

The cover may include PTFE or any other polymer. The cover may also provide more uniform drug delivery and help with more consistent and uniform compression.

In another embodiment, the elongated structure of the intraluminal device may be configured to transition between a collapsed position for delivery to a treatment site, and an expanded position in response to an opening force exerted thereon.

In another embodiment, the elongated structure of the intraluminal device may include a flexible shaft; an expandable wire mesh structure extending from the flexible shaft; and at least one actuator, connected to the expandable wire mesh structure, the actuator being configured to cooperate with the wire mesh structure to transfer angioplasty forces to a vessel obstruction. The flexible shaft may be formed in the shape of a coil, made from the same wires as the wire mesh structure. The coil may have an opening in the center for housing the actuator, which may be one or more pull wires. When a physician pulls on such wires, it may cause the wire mesh structure to expand. The wires may be connected to the wire mesh structure in a manner permitting the high forces necessary for angioplasty, as discussed earlier.

The wire mesh structure of the angioplasty device may include at least a first expandable section having a first wire arrangement pattern and at least a second expandable section having a second wire arrangement pattern different from the first wire arrangement pattern. The first expandable section of the angioplasty device may is configured to exert the angioplasty forces and wherein the second section is configured as a filter with interstices smaller than interstices in the first section. The flexible shaft of the angioplasty device may include wires that make up the expandable mesh structure.

### BRIEF DESCRIPTION OF THE DRAWINGS

The accompanying drawings, which are incorporated in and constitute a part of this specification, illustrate disclosed embodiments and, together with the description, serve to explain the disclosed embodiments.

FIG. 1 is an illustration of an exemplary intraluminal device, consistent with at least one of the disclosed embodiments in a deflated position;

FIG. 2 is an illustration of an exemplary intraluminal device, in accordance with FIG. 1, in an inflated position;

FIG. 3 is an illustration of another exemplary intraluminal device in accordance with at least one of the disclosed embodiments;

FIG. 4 is an illustration of another exemplary intraluminal device in accordance with at least one of the disclosed embodiments;

FIG. 5 is an illustration of another exemplary intraluminal device in accordance with at least one of the disclosed embodiments;

FIG. 6 is an illustration of another exemplary intraluminal device in accordance with at least one of the disclosed embodiments;

FIG. 7 is an illustration of another exemplary intraluminal device in accordance with at least one of the disclosed embodiments;

FIG. 8 is an illustration of another exemplary intraluminal device in accordance with at least one of the disclosed embodiments;

FIG. 9 is an illustration of another exemplary intraluminal device in accordance with at least one of the disclosed embodiments; and

FIG. 10 is an illustration of another exemplary intraluminal device in accordance with at least one of the disclosed embodiments; and

FIG. 11 is an illustration of another exemplary intraluminal device in accordance with at least one of the disclosed embodiments.

Annotations appearing in the figures are exemplary only, and are not restrictive of the invention as claimed.

### DETAILED DESCRIPTION

Reference will now be made in detail to the present embodiments (exemplary embodiments) of the disclosure, examples of which are illustrated in the accompanying drawings. Wherever possible, the same reference numbers will be used throughout the drawings to refer to the same or like parts.

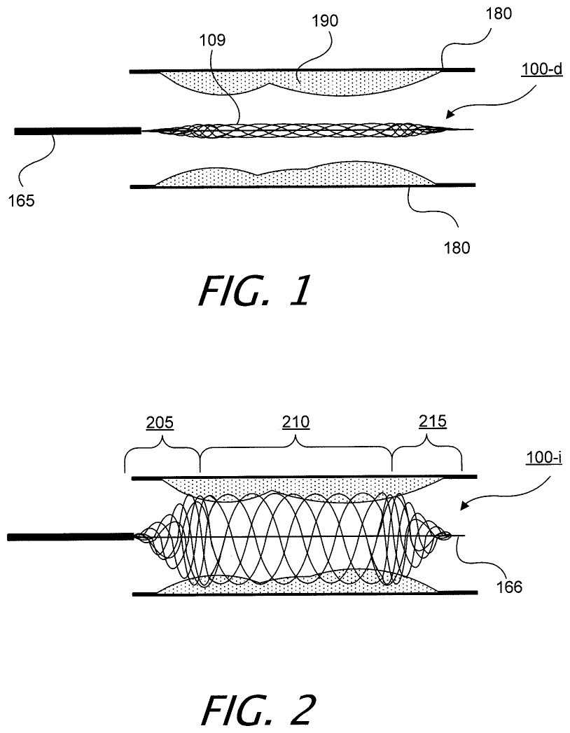

FIGS. 1 and 2 illustrate an exemplary intraluminal, adjustable non-blocking angioplasty device 100 including groups of woven, or looped wires 109 for structural support. FIG. 1 depicts device 100 in a “deflated” configuration (i.e., a compressed configuration), which is denoted in FIG. 1 with the notation “100-d.” FIG. 2 depicts device 100 in an “inflated” configuration (i.e., an expanded configuration), which is denoted in FIG. 2 with the notation “100-i.” Openings between wires 109, or groups of wires may also provide for one or more filters. In FIGS. 1 and 2, two filters are depicted: filter 205 and filter 215. The one or more filters may be provided at a distal and/or proximal end of the device, for example. As shown in FIG. 2 in an inflated configuration, the device 100 may include two filters: filter 215 located at a distal end of device 100, and filter 205 located at proximal end of device 100. In FIG. 1, for exemplary purposes only, lumen 180 is depicted with calcification 190. In addition, flexible shaft 165 is depicted.

In an exemplary form, actuator 166 may be an elongated wire that is connected to the distal end of the mesh of device 100. Actuator 166 can extend to a proximal handle that can be used to activate device 100 by pulling or releasing the actuator 166. In a further embodiment, the actuator may also be a cable or other arrangement of a plurality of wires such that when the actuator is pulled the mesh can be configured to expand. In a further embodiment, an actuator can include one or more wires of the mesh, but where the included wires are pulled back from the mesh to form a handle; in such an embodiment, when the wires in the handle are pulled, the mesh can expand. In yet a further embodiment, an actuator may be configured to maintain the position of a distal portion of the mesh when the remaining, proximal, portion of the mesh is moved forward with shaft 165. In such an embodiment, a handle may be connected to shaft 165 such that a pushing force can be imposed on shaft 165 while the actuator is stationary.

As described above, FIG. 2 illustrates an exemplary intraluminal, adjustable non-blocking angioplasty device 100 in accordance with FIG. 1, including two filters, located at a distal and proximal end of the device (filters 215 and 205, respectively), in an inflated, or expanded, position. In accordance with at least some embodiments in accordance with the present disclosure, the device 100 may provide for a high radial force zone 210 located between the two filters 205 and 215.

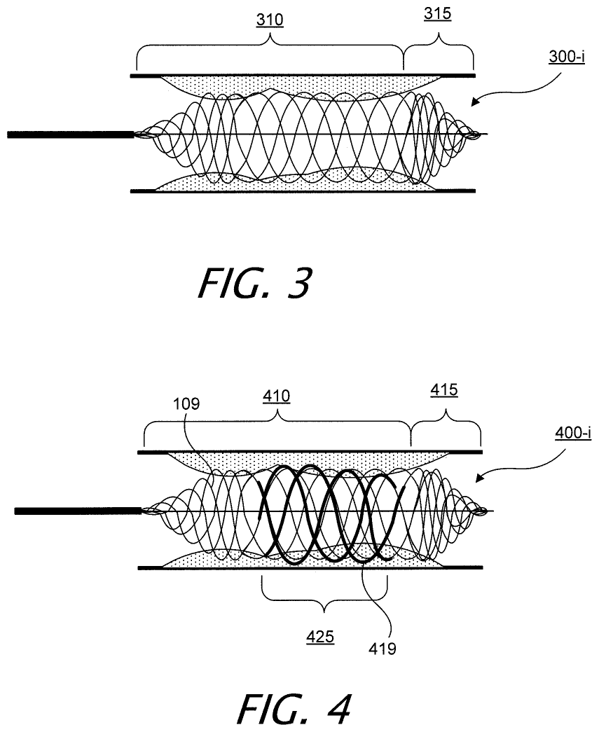

FIG. 3 illustrates another exemplary intraluminal device 300 in an inflated or expanded configuration. In this example, the adjustable non-blocking angioplasty device 300 may include, for example, one distal filter 315, which is shown in FIG. 3 in an inflated or expanded position. In accordance with at least some embodiments in accordance with the present disclosure, the device 300 may provide for a high radial force zone 310 located in an intermediate area located the distal filter 300.

FIG. 4 illustrates another exemplary intraluminal device 400. In this example, the adjustable non-blocking angioplasty device 400 may include, for example, one distal filter 415, which is shown in FIG. 4 in an inflated position. Device 400 may also include a variable mesh structure 410 providing a variable radial force. In accordance with at least some embodiments in accordance with the present disclosure, the variable mesh structure 410 may include a non-uniformity which allow for some portions of the device 400 to exert more force than other portions of the device 400. Also in accordance with at least some embodiments in accordance with the present disclosure, the device 400 may provide for both a high radial force zone 410 and a very high radial force and/or high density zone 425. For example, as depicted in FIG. 4, wires 419 may be configured to provide a greater force on the lumen (and any potential obstruction or calcification) than wires 109.

Optionally, or alternatively, the variable mesh structure in intermediate area 410 of device 400 may include a non-uniformity which allow for some portions of the device 400 to exhibit a higher density than other portions of the device 400. Accordingly, in such an embodiment, the device 400 may provide for both a high radial force zone 410 and a very high density zone 425.

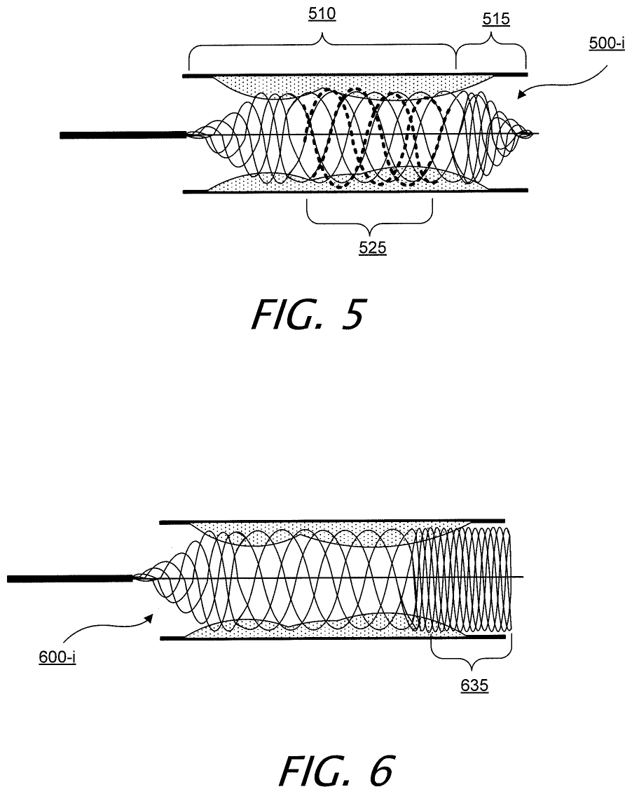

FIG. 5 illustrates yet another exemplary intraluminal device 500. In this example, the adjustable non-blocking angioplasty device 500 may include for example, one distal filter 515, which is depicted in FIG. 5 in an expanded position. In addition, device 500 may include a drug eluting region 525 located in an intermediate area 510 of the device, for example. Additionally, the entire expandable structure of device 500, or some other fraction thereof may be drug eluting. Furthermore, the mesh structure in intermediate area 510 of device 500 may provide for a high radial force zone 510.

FIG. 6 illustrates yet another exemplary intraluminal device 600. For example, the adjustable non-blocking angioplasty device 600 may include, for example, a tubular distal filter 635 or filters of other shapes. The device 600 may also be provided with a variable mesh density. It should be noted that the distal end of tubular distal filter 635 is closed, so as to provide a filtering function over the surface area of the distal end of device 635, when it is in an inflated or expanded configuration in the lumen.

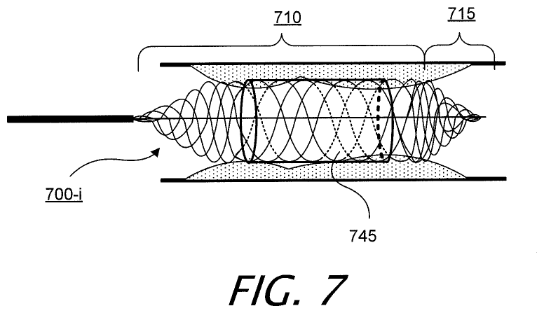

FIG. 7 illustrates yet another exemplary intraluminal device 700. For example, the adjustable non-blocking angioplasty device 700 may include, for example, a distal filter 715. In accordance with at least some embodiments in accordance with the present disclosure, the device 700 may include a covering 745 in the high radial force zone 710. The covering 745 may be drug eluting. The covering 745 may include PTFE or any other polymer. The covering 745 may also provide more uniform drug delivery and help with more consistent and uniform compression. Further still, the device 700 may be provided with a variable mesh density.

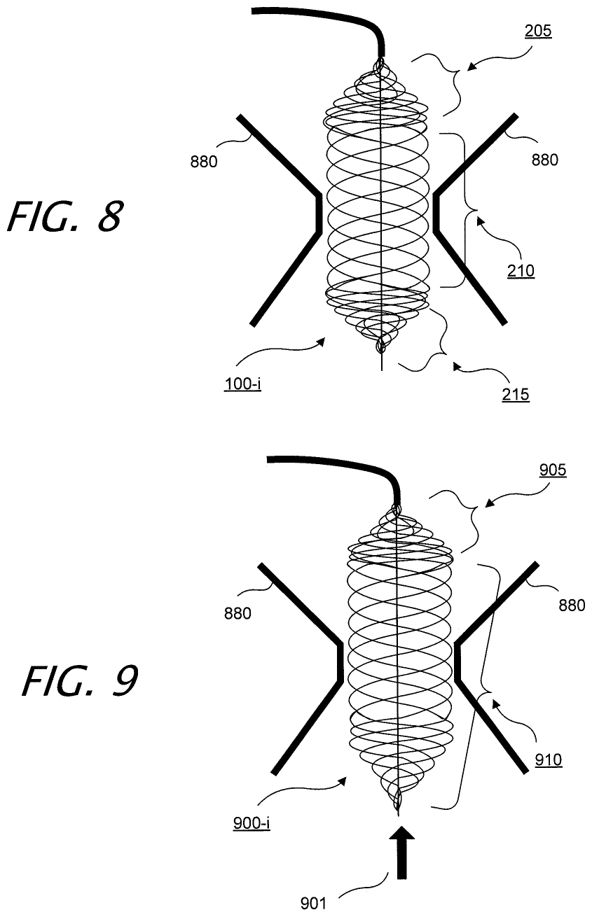

FIG. 8 illustrates exemplary intraluminal device 100 in an exemplary configuration. In this example, the adjustable non-blocking valve angioplasty device 100 may include two filters 205 and 215. Due to calcification in the heart valves 880, the device may enable blood flow through the heart as the calcification is removed. Device shapes may be tailored to the anatomy of the valve 880.

FIG. 9 illustrate yet another exemplary intraluminal device 900. In this example, the adjustable non-blocking valve angioplasty device 900 includes one filter 905, while also providing a high radial force zone 910, in the middle of the device 900, for example. Blood flow is depicted by arrow 901

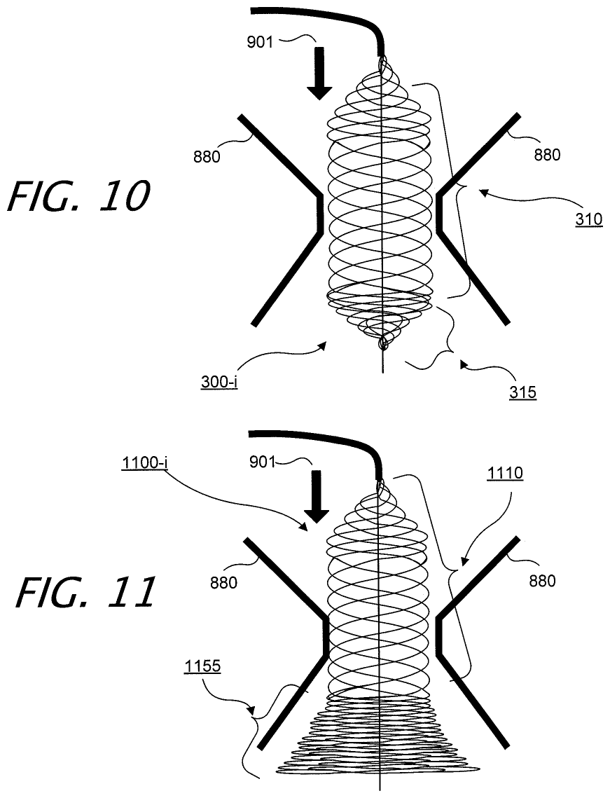

FIG. 10 illustrates exemplary intraluminal device 300 in an exemplary configuration. In this example, the adjustable non-blocking valve angioplasty device 300 includes one filter 315, while also providing a high radial force zone 310, in the middle of the device 300, for example. Again, blood flow is depicted by arrow 901.

FIG. 11 illustrate yet another exemplary intraluminal device 1100. In this example, the adjustable non-blocking valve angioplasty device 1100 may include a non-conical distal filter 1155, while also providing a high radial force zone 1110, in the middle of the device 1100, for example. The distal end of non-conical distal filter 1155 is closed, so as to provide a filtering function over the surface area of the distal end of device 1100, when it is in an inflated or expanded configuration.

The wire mesh structure of any of the angioplasty devices 100, 300, 400, 500, 600, 700, 900, and 1100 may include at least a first expandable section having a first wire arrangement pattern and at least a second expandable section having a second wire arrangement pattern different from the first wire arrangement pattern. The first expandable section of the angioplasty device (which is section 210 in device 100, section 310 in device 300, section 410 in device 400, section 510 in device 500, section 710 in device 700, section 910 in device 900, and section 1110 in device 1100) may be configured to exert the angioplasty forces and wherein the second section (which are sections 205 and 215 in device 100, section 315 in device 300, section 415 in device 400, section 515 in device 500, section 635 in device 600, section 715 in device 700, section 905 in device 900, and section 1155 in device 1100) may be configured as a filter with interstices smaller than interstices in the first section. In an embodiment, when the corresponding device is in an inflated, or expanded configuration, the average filter spacing provided by the smaller interstices in sections 205, 215, 315, 415, 515, 635, 715, 905, and 1155 may be half the size, and smaller, than the average spacing provided by the interstices in sections 210, 310, 410, 510, 710, 910, and 1110. In a further embodiment, when the corresponding device is in an inflated, or expanded configuration, the average filter spacing provided by the smaller interstices in sections 205, 215, 315, 415, 515, 635, 715, 905, and 1155 may be one-fourth the size, and smaller, than the average spacing provided by the interstices in sections 210, 310, 410, 510, 710, 910, and 1110. Moreover, in an embodiment, the flexible shaft 165 of the angioplasty device may include wires that make up the expandable mesh structure.

While illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution, of the application, which examples are to be construed as non-exclusive. Further, the steps of the disclosed methods can be modified in any manner, including by reordering steps or inserting or deleting steps. It is intended, therefore, that the specification and examples be considered as example only, with a true scope and spirit being indicated by the following claims and their full scope of equivalents.

What is claimed is:

1. An angioplasty device, comprising:
a flexible shaft;
an expandable wire mesh structure extending from the flexible shaft; and
at least one actuator, connected to the expandable wire mesh structure, the actuator being configured to cooperate with the wire mesh structure to transfer angioplasty forces to a vessel obstruction.

2. The angioplasty device of claim 1, wherein the wire mesh structure includes at least a first expandable section having a first wire arrangement pattern and at least a second expandable section having a second wire arrangement pattern different from the first wire arrangement pattern.

3. The angioplasty device of claim 2, wherein the first expandable section is configured to exert the angioplasty forces and wherein the second section is configured as a filter with interstices smaller than interstices in the first section.

4. The angioplasty device of claim 1, wherein the flexible shaft includes wires that make up the expandable mesh structure.
